(19) United States
(12) Patent Application Publication
(10) Pub. No.: US 2026/0124462 A1
(43) Pub. Date: May 7, 2026

(54) Multi-Functional Cosmetic Mirror

(71) Applicant: Shenzhen Kaiyan Medical Equipment Co., Ltd., Shenzhen (CN)

(72) Inventors: Alain Dijkstra, Amstelveen (NL); LI XIANG, Shenzhen (CN); Chen Shoufeng, Shenzhen (CN); Ye Qi, Shenzhen (CN)

(73) Assignee: Shenzhen Kaiyan Medical Equipment Co., Ltd., Shenzhen (CN)

(21) Appl. No.: 19/346,432

(22) Filed: Sep. 30, 2025

(30) Foreign Application Priority Data
Sep. 30, 2024 (CN) 2024224023269

(51) Int. Cl. A61N 5/06; A45D 44/00; A61B 5/00; A61B 5/11

(52) U.S. Cl. CPC A61N 5/0616; A45D 44/00; A61B 5/0064; A61B 5/0082; A61B 5/1114; A61B 5/441; A61B 5/7445; A45D 2044/007; A61B 5/0077; A61N 2005/0627; A61N 2005/0642; A61N 2005/0652; A61N 2005/0659

(57) ABSTRACT

The present invention relates to a multifunctional cosmetic device having dual opposite faces that combine daily cosmetic use with advanced skincare functions. A first face comprises a mirror surface integrated with a plurality of light therapy light-emitting diodes (LEDs), including infrared LEDs that emit therapeutic light within the 700-1100 nm range. Since the infrared light is invisible to the human eye, the user can simultaneously view their reflection and perform grooming or makeup application while receiving phototherapy. The opposite face comprises an ultraviolet (UV) imaging module including a UV light source, a UV-sensitive camera, and a display screen. The UV module enables real-time visualization of sunscreen coverage and hidden skin conditions not detectable under visible light. A shared rechargeable power supply and control system selectively drives the mirror-side therapy module and the UV imaging module, providing a compact, integrated skincare solution.

### TECHNICAL FIELD

The present invention relates to the field of cosmetic and skincare devices, and more particularly to a multifunctional cosmetic mirror that integrates a mirror surface with auxiliary modules for skin imaging and light therapy. Specifically, the invention concerns a dual-faced device having on one side a mirror integrated with invisible infrared light therapy elements, and on the opposite side an ultraviolet imaging system comprising a UV light source, a UV-sensitive camera, and a display screen for assessing sunscreen application and skin conditions.

### BACKGROUND

The appearance and health of facial skin are central concerns for many individuals engaged in daily grooming and cosmetic care. A key problem faced by users is the difficulty of accurately evaluating their skin condition and maintaining effective skincare routines with limited tools. Ordinary mirrors only provide surface-level reflection, preventing users from detecting deeper skin issues such as uneven sunscreen application, underlying pigmentation, or early signs of aging. Similarly, skin treatment devices are often bulky, single-purpose tools that must be stored separately, making them inconvenient for everyday use.

Traditionally, individuals have relied on basic handheld or table-top mirrors for grooming and cosmetic application. While some mirrors incorporate magnification, they are still unable to provide deeper diagnostic insights. For skincare treatment, dermatological clinics have long employed specialized phototherapy equipment; however, such systems are costly, non-portable, and inaccessible for routine daily use at home.

To address consumer demand for convenience, the market has introduced a range of home-use devices. These include handheld UV inspection devices that reveal sunscreen coverage or sun damage, and portable light therapy instruments that use red, blue, or infrared light to promote skin rejuvenation or acne treatment. Some modern vanity mirrors also incorporate fill lighting for improved visibility. While useful, these solutions remain fragmented, requiring multiple devices for imaging, inspection, and therapy. As a result, users face increased expense, storage difficulties, and disruption in daily routines.

Accordingly, a clear gap exists in the market: there is no single compact system that combines the everyday utility of a mirror with advanced ultraviolet inspection and therapeutic light treatment. Users are forced to choose between basic mirrors with limited diagnostic functions or specialized therapy devices that lack daily usability as cosmetic mirrors.

The present invention addresses this gap by providing a multifunctional cosmetic mirror that integrates the reflective function of a makeup mirror, the diagnostic capability of ultraviolet imaging, and the therapeutic benefits of phototherapy in a single, user-friendly structure. Through this integration, the device allows users to seamlessly transition from daily grooming to skin analysis and treatment, thereby improving convenience, increasing utilization, and enhancing the overall effectiveness of personal skincare routines.

### OBJECTS OF THE INVENTION

Some of the objects of the invention are as follows:

An object of the present invention is to provide a multifunctional cosmetic mirror that combines the everyday functionality of a mirror with advanced imaging and phototherapy capabilities.

Another object of the present invention is to provide a cosmetic mirror with an ultraviolet (UV) imaging unit that enables accurate assessment of sunscreen application and detection of skin conditions not visible under ordinary light.

Another object of the present invention is to provide a cosmetic mirror with a phototherapy module incorporating infrared light-emitting elements, allowing therapeutic treatment to be performed simultaneously with makeup application or grooming.

Another object of the present invention is to provide a cosmetic mirror with light therapy elements that operate in the infrared spectrum (700-1100 nm), thereby delivering therapeutic benefits while remaining invisible to the human eye and without disturbing normal cosmetic use.

Another object of the present invention is to provide a cosmetic device with circumferential fill-light modules to improve visibility during both visible light imaging and ultraviolet imaging modes.

Another object of the present invention is to provide a multifunctional cosmetic mirror with modular imaging units, including a visible light camera and a UV-sensitive camera, that may be alternately activated according to the user's needs.

Another object of the present invention is to provide a cosmetic mirror with an integrated power supply and control unit, enabling selective activation of the visible imaging unit, the UV imaging unit, or the phototherapy module.

Another object of the present invention is to provide a cosmetic mirror with interchangeable mirror configurations, including plane mirrors and concave mirrors, for enhanced user convenience.

Another object of the present invention is to provide a compact, dual-faced cosmetic mirror that eliminates the need for separate mirrors, UV inspection devices, and standalone phototherapy instruments, thereby reducing cost, saving space, and improving user experience.

Another object of the present invention is to provide a multifunctional cosmetic mirror suitable for skincare, cosmetic, and dermatological applications, ensuring reliable daily use, preventive skin monitoring, and therapeutic treatment in a single integrated system.

### SUMMARY OF THE INVENTION

According to a first aspect of the present invention, a multifunctional cosmetic mirror device is provided. The device comprising: a mirror frame; a mirror surface configured for use as a cosmetic mirror mounted on a first side of the mirror frame; and at least one module mounted on a second side of the mirror frame; wherein the at least one module is selected from an imaging module configured to analyze a skin condition of a user, or a phototherapy module configured to apply therapeutic light to the user's skin.

In one embodiment of the invention, the device further comprises a control unit configured to selectively activate the imaging module or the phototherapy module.

In one embodiment of the invention, the at least one module comprises both the imaging module and the phototherapy module.

In one embodiment of the invention, the device further comprising: a distance sensor configured to measure a distance between the skin and the at least one module.

In one embodiment of the invention, the device further comprising: a fill-light module arranged circumferentially around the mirror surface.

In one embodiment of the invention, the phototherapy module is provided on the first side of the mirror frame, having the mirror surface.

In one embodiment of the invention, the phototherapy module is arranged on a side of the mirror surface, behind the mirror surface, or on or adjacent to the mirror surface.

In one embodiment of the invention, the phototherapy module is configured to irradiate infrared light towards the user while the mirror surface is used for cosmetic viewing.

According to a second aspect of the present invention, a multifunctional cosmetic mirror device is provided. The device comprising: a frame having a first side and a second side; a mirror surface provided on the first side of the frame; with a first display surface on the first side of the frame to display an image of a user; a phototherapy module comprising an infrared light-emitting assembly associated with the first side of the frame and configured to irradiate the user's skin with invisible infrared light while the mirror surface reflects the user's visible image; an ultraviolet (UV) imaging unit disposed on the second side of the frame; and a activation mechanism arranged in the frame and coupled to a control circuit, the activation mechanism being configured to selectively activate either the visible light imaging unit and the infrared light-emitting assembly when the first side faces the user, or the UV imaging unit when the second side faces the user.

In one embodiment of the invention, the activation mechanism comprises a mechanical limit switch actuated by rotation of the frame.

In one embodiment of the invention, the activation mechanism comprises an orientation sensor configured to detect whether the first side or the second side of the frame faces the user.

In one embodiment of the invention, the infrared light-emitting assembly comprises at least one infrared LED operating within a wavelength range of 700 nm to 1100 nm.

In one embodiment of the invention, the control circuit is configured to prevent simultaneous activation of the UV imaging unit and the infrared light-emitting assembly.

In one embodiment of the invention, the UV imaging unit comprises a UV lamp and a UV-sensitive camera configured to capture and display UV features of the user's skin.

In one embodiment of the invention, the displaying of the image of the user on the first display surface and the infrared light-emitting assembly are configured to operate simultaneously when the first side faces the user.

According to a third aspect of the present invention, a method of operating a multifunctional cosmetic mirror device is provided. The method comprising: providing a mirror surface on a first side of a frame; activating an infrared light-emitting assembly disposed in association with the mirror surface; simultaneously reflecting a visible image of a user by the mirror surface and irradiating the user's skin with invisible infrared light emitted from the infrared light-emitting assembly; detecting, by a distance sensor, a spacing between the user and the mirror surface; and controlling, by a control circuit coupled to the infrared light-emitting assembly and the distance sensor, operation of the infrared light-emitting assembly such that infrared irradiation is enabled or disabled based on whether the detected spacing falls within a predetermined safe range.

In one embodiment of the invention, controlling the operation of the infrared light-emitting assembly comprises disabling the infrared light when the detected spacing is below a safe minimum distance.

In one embodiment of the invention, the infrared light-emitting assembly comprises at least one infrared LED operating within a wavelength range of 700 nm to 1100 nm.

In one embodiment of the invention, the method further comprising: rotating the frame such that a second side having a UV imaging unit faces the user, and automatically disabling the infrared light-emitting assembly while enabling the UV imaging unit.

In one embodiment of the invention, the method further comprising: enabling simultaneous activation of the mirror surface for visible reflection and the infrared light-emitting assembly for therapeutic irradiation during cosmetic application.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “flexible substrate” refers to a base material that supports the placement and interconnection of electrical components, while allowing the overall structure to bend or conform to non-planar surfaces. Examples include polyimide film, thermoplastic elastomers, and silicone-based sheets.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 illustrates a perspective view of a first surface of a multifunctional cosmetic mirror, in accordance with an embodiment of the present invention.

FIG. 2 illustrates a perspective view of a second surface of a multifunctional cosmetic mirror, in accordance with an embodiment of the present invention.

FIG. 3 illustrates an exploded view of the multifunctional cosmetic mirror, in accordance with an embodiment of the present invention.

FIG. 4 illustrates an exploded view of a second light source of the multifunctional cosmetic mirror, in accordance with an embodiment of the present invention.

FIG. 5 illustrates a cross-sectional view of the multifunctional cosmetic mirror with points ‘A’ and ‘B’, in accordance with an embodiment of the present invention.

FIG. 6 illustrates an enlarged view of point ‘A’ shown in FIG. 5, in accordance with an embodiment of the present invention.

FIG. 7 illustrates an enlarged view of point ‘B’ shown in FIG. 5, in accordance with an embodiment of the present invention.

FIG. 8 illustrates a perspective view of a first surface of an alternate configuration of a multifunctional cosmetic mirror, in accordance with an embodiment of the present invention.

FIG. 9 illustrates a perspective view of a second surface of the alternate configuration of the multifunctional cosmetic mirror, in accordance with an embodiment of the present invention.

FIG. 10 illustrates an exploded view of the first surface of the alternate configuration of the multifunctional cosmetic mirror, in accordance with an embodiment of the present invention.

FIG. 11 illustrates an exploded view of the second surface of the alternate configuration of the multifunctional cosmetic mirror, in accordance with an embodiment of the present invention.

FIG. 12 illustrates a cross-sectional view of the multifunctional cosmetic mirror with a point ‘C’, in accordance with an embodiment of the present invention.

FIG. 13 illustrates an enlarged view of point ‘C’ shown in FIG. 12, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention will now be described in detail with reference to the accompanying drawings, wherein like reference numerals designate like or corresponding components throughout. The embodiments illustrate a multifunctional cosmetic device that integrates a mirror surface, a phototherapy module, and an ultraviolet (UV) imaging system into a single compact housing. The device is configured with two opposite functional faces, each adapted to provide distinct cosmetic and therapeutic benefits while sharing common structural and control elements.

Embodiments of the present invention disclose a multifunctional cosmetic mirror device configured to combine conventional cosmetic mirror functionality with imaging and phototherapy capabilities for enhanced skin analysis and treatment. The cosmetic mirror device comprises a mirror frame having at least a first side and a second side. A mirror surface is mounted on the first side of the frame and is configured for use as a cosmetic mirror by a user. At least one functional module is mounted on the frame, the module being selected from an imaging module configured to analyze a skin condition of the user and a phototherapy module configured to apply therapeutic light to the skin. In certain embodiments, both the imaging module and the phototherapy module are provided within the same device, and a control unit is arranged to selectively activate one or both modules depending on the operational mode.

In some embodiments, the second side of the mirror frame is provided with both the ultraviolet (UV) imaging module and the phototherapy module. In such configurations, the UV imaging module may be employed to analyze subsurface skin features under UV illumination, while the phototherapy module may deliver therapeutic light such as red, blue, or infrared wavelengths to the skin. The arrangement of both modules on the second side of the frame allows the device to function as a dedicated diagnostic and therapeutic unit when rotated, thereby extending its versatility beyond conventional mirror use and providing the user with comprehensive skincare analysis and treatment capabilities within a single device.

In one embodiment, the phototherapy module is arranged on the same side of the frame as the mirror surface. The phototherapy module may be disposed at the side of the mirror surface, behind the mirror surface, or directly on or adjacent to the mirror surface. The phototherapy module is configured to irradiate UV light, infrared light, blue light, or red light. In a preferred embodiment, the phototherapy module comprises an infrared light-emitting assembly configured to irradiate invisible infrared light toward the user while the mirror surface reflects a visible image. This arrangement enables a user to simultaneously view their reflection in the mirror while receiving phototherapy treatment without visual interference. The infrared light-emitting assembly may include one or more infrared light-emitting diodes (LEDs) operating in the wavelength range of approximately 700 nm to 1100 nm.

A distance sensor may be provided in proximity to the mirror surface to detect the spacing between the user and the mirror. The distance sensor is coupled to the control circuit such that operation of the phototherapy module is enabled or disabled based on whether the detected spacing falls within a predetermined safe range. This ensures safe and reliable delivery of phototherapy by preventing overexposure when the user's face is too close to the mirror surface.

In certain embodiments, the phototherapy module is configured to emit light in the far-infrared spectrum, which is particularly effective for promoting skin rejuvenation and stimulating deeper tissue layers while not interfering with the user's visibility during mirror use. During conventional operation of the mirror, far-infrared light is preferably applied so that the user may simultaneously perform cosmetic application and receive therapeutic benefits.

The device may further be configured to dynamically adjust the spectral range and intensity of emitted radiation based on the spacing between the user and the mirror, as determined by the distance sensor. For example, when the user is positioned closer to the mirror, the control circuit may automatically reduce the emission intensity or switch the emission to near-infrared wavelengths to ensure safe exposure. Conversely, when the user is located at a greater distance, the control circuit may allow higher intensity or switch to far-infrared output to maintain therapeutic efficacy. This adaptive control of wavelength and intensity provides a personalized and safe phototherapy experience while maximizing treatment effectiveness.

In another embodiment, the multifunctional cosmetic device is implemented as a dual-sided frame. The first side of the frame comprises the mirror surface, a visible light imaging unit, and the phototherapy module, while the second side of the frame comprises an ultraviolet (UV) imaging unit. The UV imaging unit may include a UV lamp and a UV-sensitive camera to capture and display UV images of the user's skin, thereby allowing detection of hidden blemishes, pigmentation, or sun damage. An activation mechanism is integrated within the frame and coupled to the control circuit to selectively activate either the visible light imaging unit and the phototherapy module when the first side faces the user, or the UV imaging unit when the second side faces the user. The activation mechanism may be a mechanical limit switch actuated by rotation of the frame, or an orientation sensor that automatically detects which side of the frame is oriented toward the user. The control circuit may further be configured to prevent simultaneous activation of the UV imaging unit and the phototherapy module to ensure operational safety.

A fill-light module may optionally be arranged circumferentially around the mirror surface to provide uniform illumination for cosmetic application or imaging purposes. In certain embodiments, the visible light imaging unit and the phototherapy module may operate simultaneously, enabling both visual reflection of the user and concurrent phototherapy treatment. The device may further be configured with a user interface to control light intensity, duration of treatment, and switching between imaging modes.

In another embodiment of the present invention, a method of operating a multifunctional cosmetic mirror device is provided. The method enables simultaneous cosmetic mirror use and phototherapy treatment under safe operating conditions.

In one embodiment, the method comprises providing a mirror surface on a first side of a frame, the mirror surface being configured to reflect a visible image of a user. An infrared light-emitting assembly is disposed in association with the mirror surface and is activated during operation. When the user positions their face in front of the mirror, the mirror surface reflects the visible image of the user, while at the same time, the phototherapy module provides phototherapy to the user's skin. This arrangement enables the user to view their reflection in real time without visual interference, while receiving therapeutic exposure to infrared wavelengths. The phototherapy module is configured to irradiate red light, blue light, or infrared light. In a preferred embodiment, the phototherapy module is configured to irradiate infrared light, to enable a user to simultaneously use the mirror as well as receive phototherapy.

The method further comprises detecting, by means of a distance sensor, a spacing between the user and the mirror surface. The distance sensor generates a distance signal that is communicated to a control circuit. The control circuit is operatively coupled to the infrared light-emitting assembly and regulates its operation based on the detected spacing. When the user is located within a predetermined safe distance range, the control circuit permits activation of the infrared assembly. If the detected spacing falls below a safe minimum threshold, the control circuit disables the infrared emission to prevent overexposure. In this way, user safety is maintained while still allowing efficient phototherapy.

In certain embodiments, the method includes controlling specific operational parameters of the infrared assembly, such as the irradiation wavelength, intensity, or duration of exposure. The infrared light-emitting assembly may comprise one or more infrared LEDs configured to emit within the wavelength range of approximately 700 nm to 1100 nm, thereby targeting skin layers most suitable for cosmetic and therapeutic treatment.

In another embodiment, the method further comprises rotating the frame such that a second side having an ultraviolet (UV) imaging unit faces the user. In this position, the control circuit automatically disables the infrared assembly and enables the UV imaging unit. The UV imaging unit may capture and display UV images of the user's skin, allowing analysis of subsurface pigmentation, blemishes, or sun damage. Switching between the two operational modes: infrared phototherapy and UV imaging, is thus achieved seamlessly based on the orientation of the frame, thereby simplifying user interaction.

The multifunctional cosmetic device described herein offers several advantages over conventional cosmetic mirrors and standalone imaging or therapy devices. It integrates mirror, imaging, and phototherapy functionalities into a single compact unit, thereby reducing cost and space requirements. By providing simultaneous visible reflection and invisible infrared therapy, the device supports convenient cosmetic application while delivering skin treatment. The inclusion of UV imaging on a second side of the frame extends the functionality to diagnostic use, enabling users to assess underlying skin conditions. Safety features such as distance sensing and exclusive activation control further enhance user confidence and reliability of operation.

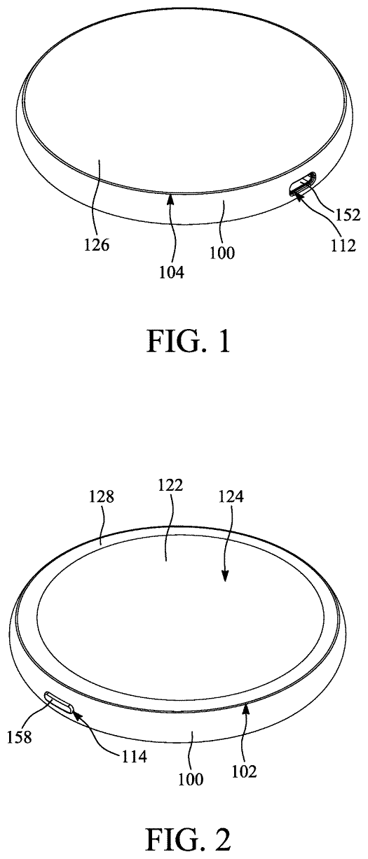

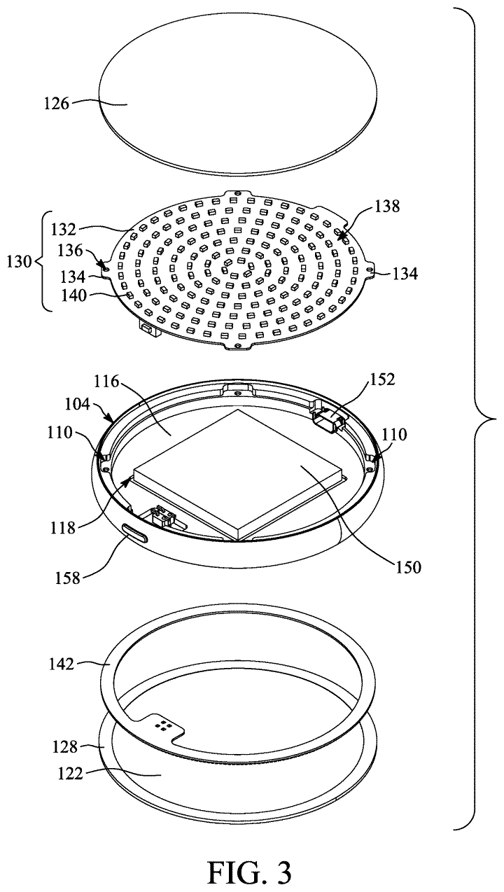

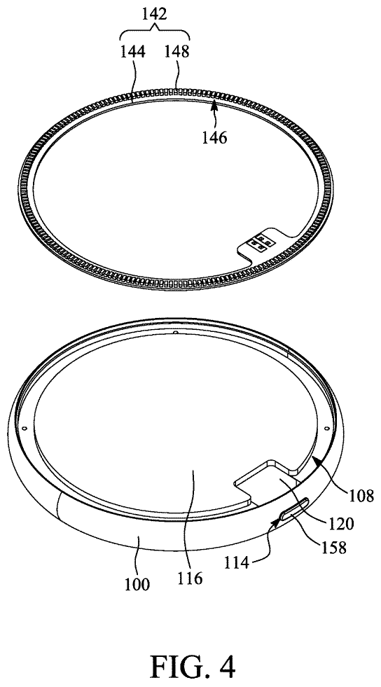

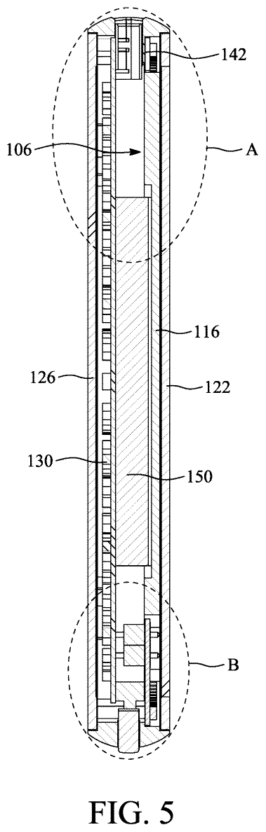

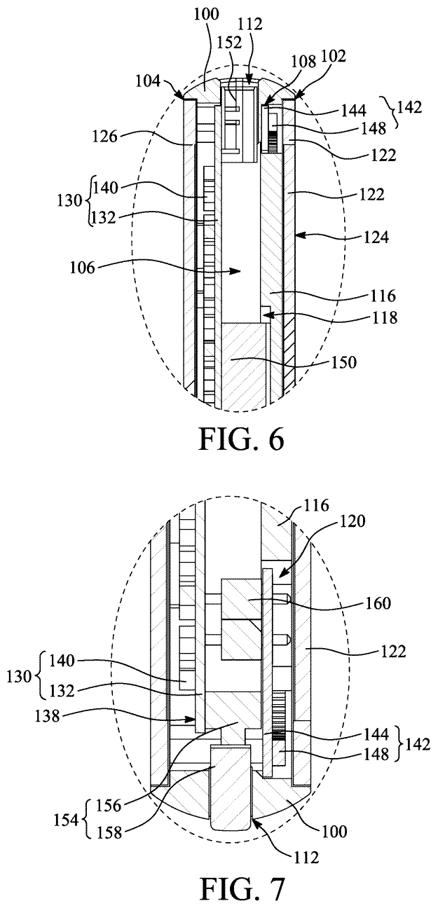

Referring to FIGS. 1 to 7, a multifunctional cosmetic mirror device is disclosed. The cosmetic mirror device comprises a housing 100, a lens 122, a light-transmitting plate 126, and a phototherapy module 130. The housing 100 is formed with a first surface 102 and a second surface 104 disposed opposite to one another.

The lens 122 is mounted on the first surface 102 and is provided with a plane mirror 124 facing outward, away from the second surface 104. The light-transmitting plate 126 is arranged at the second surface 104 and, together with the housing 100, defines a mounting cavity 106. The phototherapy module 130 is positioned within the mounting cavity 106 in correspondence with the light-transmitting plate 126, such that light emitted from the phototherapy module 130 is transmitted through the light-transmitting plate 126 to the exterior of the device.

The phototherapy module 130 includes at least one light-emitting element 140 configured to generate light of a predetermined wavelength. In operation, the light emitted by the light-emitting element 140 passes outward through the light-transmitting plate 126 and is applied to the skin of the user for therapeutic treatment. The light-emitting elements 140 may be configured to emit one or more types of light, including but not limited to red light, yellow light, blue light, white light, or purple light. Multiple types of light-emitting elements 140 may be selectively incorporated depending on the required therapeutic effect.

The lens 122 additionally functions as a cosmetic mirror for the user. When phototherapy is not in use, the multifunctional phototherapy device operates as a conventional makeup mirror, thereby eliminating the need for the user to purchase a separate mirror.

The housing 100 may be fabricated from glass, metal, plastic, or other suitable materials having adequate structural and cosmetic properties. In the embodiment shown, the multifunctional phototherapy device provides the lens 122 on one side of the housing 100 and the light-transmitting plate 126 with the phototherapy module 130 on the opposite side, thereby integrating reflective and therapeutic functionalities into a single compact device. In one embodiment of the present utility model, the phototherapy module 130 is disposed on one side of the multifunctional device. When light therapy is required, the user may operate the phototherapy module 130 to perform treatment. When light therapy is not needed, the multifunctional light therapy device may instead be used as a conventional cosmetic mirror for daily grooming. This dual functionality enhances the utilization rate of the device, avoids the situation where the light therapy device must be stored separately when not in use, and reduces the user's need to repeatedly organize, retrieve, and set up the device. Accordingly, the invention provides improved convenience and user experience.

Referring to FIGS. 1, 3, 5, and 6, in an embodiment, the phototherapy module 130 comprises a mounting plate 132 and a plurality of light-emitting elements 140. The mounting plate 132 is arranged within the mounting cavity 106 of the housing 100, with a first mounting surface 138 facing the light-transmitting plate 126. The plurality of light-emitting elements 140 is mounted on the first mounting surface 138. The mounting plate 132 is sized to substantially correspond to the dimensions of the second surface 104, thereby maximizing the area available for mounting light-emitting elements 140 and improving therapeutic coverage for the user.

The plurality of light-emitting elements 140 may be configured to emit light of the same wavelength, or may include a combination of different types of elements. For example, the light-emitting elements 140 comprise one or more infrared light-emitting elements, selected according to the intended therapeutic application.

In a preferred embodiment, the phototherapy module employs infrared light-emitting diodes (LEDs) that emit therapeutic light within a wavelength range substantially invisible to the human eye, for example, between approximately 700 nm and 1100 nm. Because the emitted light is outside the visible spectrum, the user is not distracted or disturbed by glare during use of the mirror. This arrangement allows the user to comfortably perform grooming or cosmetic application while simultaneously receiving phototherapy without risk of visual discomfort or harm to the eyes.

In certain embodiments, the phototherapy module is configured to emit light in the far-infrared spectrum, which is particularly effective for promoting skin rejuvenation and stimulating deeper tissue layers while not interfering with the user's visibility during mirror use. During conventional operation of the mirror, far-infrared light is preferably applied so that the user may simultaneously perform cosmetic application and receive therapeutic benefits.

The use of invisible infrared light further provides both safety and functional benefits. Unlike certain high-intensity visible light sources that may cause eye strain, the infrared emission penetrates into the skin to stimulate beneficial biological effects such as improved blood circulation, enhanced collagen synthesis, and repair of skin tissues. Since the therapy light remains unseen to the naked eye, the multifunctional cosmetic mirror retains the appearance and usability of a conventional mirror during operation, thereby improving user acceptance and practicality in everyday routines.

The infrared LEDs may be implemented in different structural configurations to optimize therapeutic coverage. In some designs, the LEDs may be aggregated in clusters to concentrate treatment on specific facial areas or distributed uniformly across a mounting plate to provide consistent irradiation. Alternatively, the LEDs may be arranged circumferentially in a ring pattern surrounding the mirror surface, ensuring balanced illumination while keeping the central reflective region unobstructed. Combinations of clustered, distributed, and ring-shaped arrangements may also be used to achieve both targeted and comprehensive therapeutic effects. Alternatively, the infrared LEDs are positioned behind the mirror surface.

In an embodiment, the mounting plate 132 is formed as a printed circuit board (PCB) on which the light-emitting elements 140 are directly mounted. In alternative embodiments, the mounting plate 132 may be a plastic substrate in combination with a PCB to provide electrical and structural support for the light-emitting elements 140.

In an embodiment, the multifunctional light therapy device further comprises a battery 150 and a charging component 152. The charging component 152 and the phototherapy module 130 are both electrically connected to the battery 150. Accordingly, the charging component 152 may replenish the battery 150, while the battery 150 supplies operating power to the phototherapy module 130. This arrangement eliminates the need for continuous external power during use, thereby enhancing portability and convenience for the user.

In an embodiment, the housing 100 includes a partition 116 disposed between the first surface 102 and the second surface 104, such that the partition 116, together with the light-transmitting plate 126, defines the mounting cavity 106. The side wall of the housing 100 surrounding the partition 116 is formed annularly, with its opposite ends defining the first surface 102 and the second surface 104. The provision of partition 116 enhances the overall structural strength of the housing 100, improves the support for the lens 122, and thereby increases the reliability and durability of the multifunctional light therapy device.

In an embodiment, the battery 150 is disposed within the mounting cavity 106, thereby facilitating centralized assembly of the battery 150 together with the phototherapy module 130. In such an arrangement, the partition 116 is positioned adjacent to the lens 122. The partition 116 may be formed with a relief groove 118, within which the battery 150 is accommodated. By sharing space in the thickness direction of the partition 116, the relief groove 118 allows efficient positioning of the battery 150 without excessively increasing the overall thickness of the multifunctional light therapy device, while also enabling larger battery capacity and simplified installation.

In an embodiment, the peripheral wall of the mounting cavity 106 is provided with a plurality of positioning grooves 110. The mounting plate 132 includes a plurality of positioning portions 134 disposed around its periphery. The positioning portions 134 are configured to engage with the corresponding positioning grooves 110, thereby securing the mounting plate 132 in position and ensuring stable alignment within the housing 100.

In an embodiment, the provision of the positioning portions 134 facilitates accurate installation of the mounting plate 132 within the mounting cavity 106. The positioning portions 134 are received within the corresponding positioning grooves 110, thereby limiting displacement and enhancing the installation stability of the mounting plate 132.

In an embodiment, each positioning portion 134 is formed with a through hole 136, and the bottom wall of the corresponding positioning groove 110 is provided with a screw hole. The multifunctional phototherapy device further comprises fastening screws which extend through the through holes 136 and are threaded into the screw holes. In this manner, the positioning portions 134 are locked within the positioning grooves 110, thereby firmly securing the mounting plate 132 and further improving structural stability.

Referring to FIGS. 2, 4, 5, 6, and 7, in an embodiment, the side of the multifunctional phototherapy device corresponding to the lens 122 is further provided with a light-transmitting portion 128, within which a fill light assembly 142 is mounted. The fill light assembly 142 is configured to emit illumination through the light-transmitting portion 128 to enhance the visibility of facial details when the user applies skincare products or makeup. The light-transmitting portion 128 may be formed integrally with the lens 122 (for example, when the lens 122 is made of plastic or glass), or may be separately attached to the periphery of the lens 122 (for example, when the lens 122 is formed of metal).

In an embodiment, the fill light assembly 142 may alternatively employ ultraviolet (UV) light sources in place of or in addition to conventional visible light lamp beads. When configured with UV-emitting lamp beads, the annular arrangement provides uniform ultraviolet illumination across the user's facial surface. Such a configuration is particularly advantageous for sunscreen evaluation, pigmentation detection, and other skin condition assessments that rely on UV reflectance properties. The uniform distribution of UV lamp beads around the light-transmitting portion 128 ensures consistent illumination, thereby enhancing imaging accuracy.

To complement the UV illumination, a UV-sensitive camera may be mounted on the same side of the multifunctional phototherapy device corresponding to the light-transmitting portion 128. During operation, the UV camera captures reflected ultraviolet light from the user's face and generates corresponding image data, which can be processed and displayed on a screen or display module.

In an embodiment of the present invention, the UV light source emits light having a peak wavelength or otherwise sufficient intensity in the range of about 290 nm to about 320 nm (e.g., in the UVB range). The UV-sensitive camera is able to capture an absolute measurement of the amount of UV light that is reflected or absorbed by the target skin surface. Optionally, UV light source emits light at two wavelength bands. For example, a UV light source may comprise a first UV-emitting lamp beads that is configured to emit light in the UVB range (e.g., from about 290 nm to about 320 nm) and a second UV-emitting lamp beads that is configured to emit light in the UVA range (e.g., from about 320 nm to about 400 nm). A UV light source capable of emitting light in the UVA and UVA bands separately may allow a user to determine the efficacy of a particular sunblock under different UV light conditions.

In an embodiment, the fill light assembly 142 comprises an annular driver board 144 and a plurality of lamp beads 148. The annular driver board 144 defines a second mounting surface 146, on which the plurality of lamp beads 148 are arranged circumferentially. The annular configuration of the driver board 144 facilitates straightforward processing and assembly, while the circumferential distribution of lamp beads 148 provides uniform illumination directed toward the light-transmitting portion 128. The battery 150 is electrically connected to the annular driver board 144, thereby supplying power to the fill light assembly 142.

In an embodiment, the housing 100 is formed with an annular groove 108 into which the annular driver board 144 is received. This arrangement simplifies the positioning and installation of the fill light assembly 142 while preventing the lamp beads 148 from abutting directly against the light-transmitting portion 128, thereby ensuring reliable assembly and operation.

In an embodiment, the light-transmitting plate 126 is formed as a transparent plate having a frosted surface, or alternatively as a translucent white plate. The frosted or translucent configuration enhances light diffusion, improves uniformity of illumination, and prevents the user from directly viewing the internal structure of the device, thereby providing a more aesthetically pleasing appearance.

In an embodiment, the housing 100 is further provided with a first through hole 112 disposed between the first surface 102 and the second surface 104. The charging component 152 is arranged within the first through hole 112 and may comprise a charging connector, such as a female connector or a Micro-USB interface. This structure offers simplicity, low production cost, and reliable charging capability. In alternative embodiments, the charging component 152 may be implemented as a wireless charging coil to enable contactless charging of the battery 150.

In an embodiment, the partition 116 is formed with a second through hole 120, and the side wall of the housing 100 is provided with a corresponding third through hole 114 aligned with the second through hole 120. The multifunctional phototherapy device further comprises a key assembly 154. The key assembly 154 is disposed within the second through hole 120 and partially exposed through the third through hole 114 of the housing 100. The key assembly 154 is electrically connected to the phototherapy module 130 for operation control.

Specifically, the key assembly 154 includes at least one key switch 156 and a corresponding keycap 158. The keycap 158 is positioned within the third through hole 114, while the key switch 156 is arranged within the second through hole 120. This structural arrangement enables the available space defined by the second through hole 120 to be effectively utilized for accommodating the key assembly 154, thereby improving the compactness of the multifunctional phototherapy device while maintaining reliable operability.

Alternatively, the multifunctional cosmetic mirror device further includes a connector 160 disposed at the second through hole 120. The connector 160 provides electrical interconnection between the mounting plate 132 of the phototherapy module and the annular driver board 144 of the fill light assembly 142, thereby ensuring stable signal transmission and power distribution between the two assemblies.

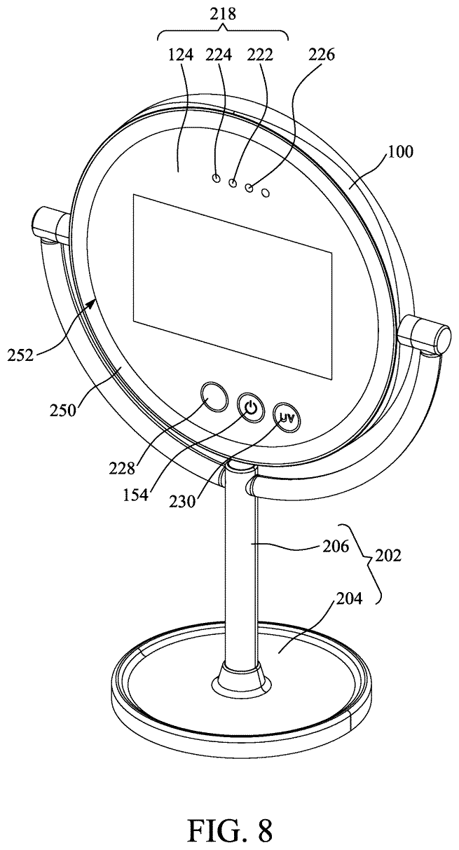

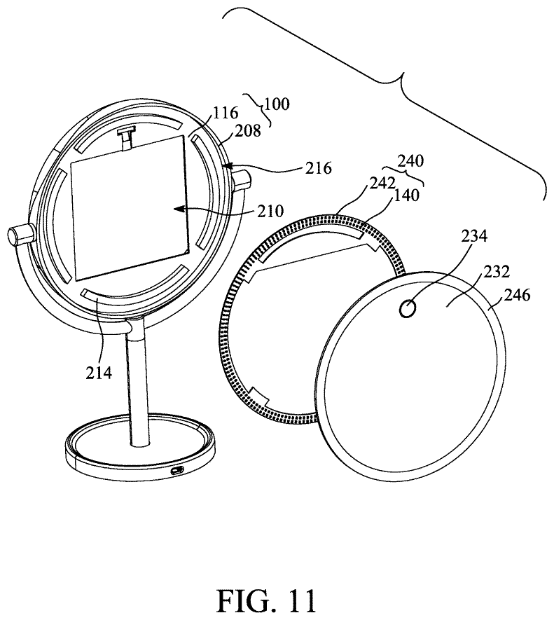

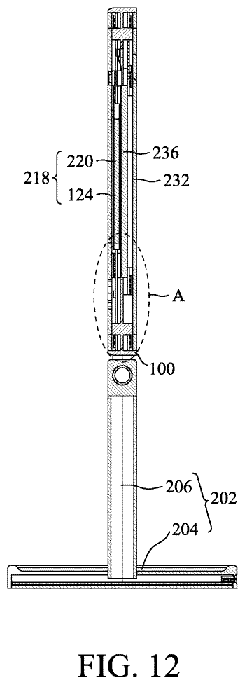

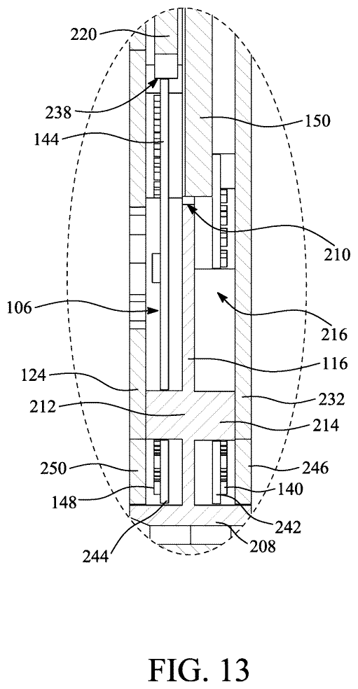

Referring to FIGS. 8 to 13, in an embodiment, an alternate configuration of a multifunctional cosmetic mirror is provided. The multifunctional cosmetic mirror comprises a housing 100, a first display surface 232, a second display module 218, and a power supply assembly 236.

The first display surface 232 is mounted on one side of the housing 100 and is configured to display visible light imaging of the user's face. The second display module 218 is mounted on the opposite side of the housing 100 and is configured to display ultraviolet (UV) imaging. The power supply assembly 236 is arranged within the housing 100, positioned between the first display surface 232 and the second display module 218, and is electrically connected to the second display module 218 to provide operating power.

In use, the first display surface 232 allows the user to directly view and assess the condition of their facial skin under visible light, which is particularly suitable for daily cosmetic and grooming applications. The second display module 218 is employed when the user applies sunscreen. After sunscreen application, the user may activate the second display module 218 to display an ultraviolet image of the face. The ultraviolet reflection allows the user to evaluate whether the sunscreen has been evenly applied across the skin. Additionally, the second display module 218 enables the observation of skin conditions that are not readily visible under standard lighting, such as hidden pigmentation or uneven texture.

Moreover, the technical solution of the present utility model achieves multi-functionality by providing, on one side of the housing 100, the first display surface 232 for visible light imaging, and on the opposite side, the second display module 218 for ultraviolet imaging. Through this dual arrangement, the multifunctional cosmetic mirror allows the user to selectively observe their facial skin under visible light or ultraviolet imaging conditions. This not only facilitates routine cosmetic use but also supports skin analysis and evaluation of sunscreen application. The multifunctional structure enriches the available functions of the mirror, overcomes the limitations of traditional cosmetic mirrors that provide only single-function reflection, and satisfies diverse user needs in both cosmetic and skincare applications.

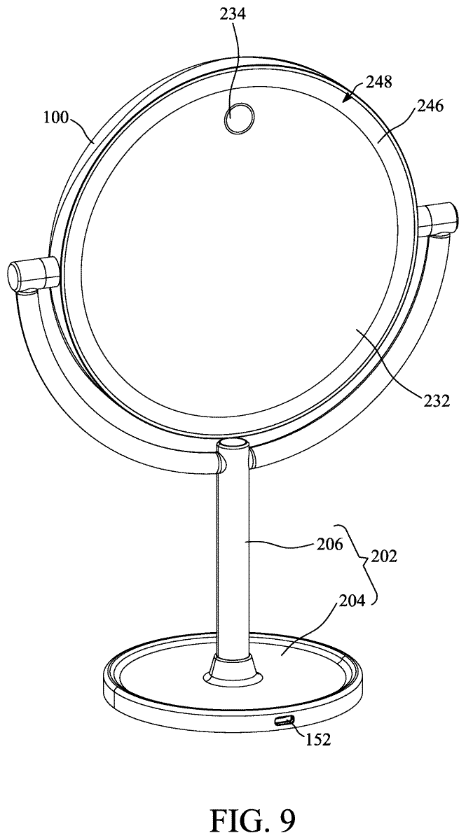

Referring to FIGS. 9, 11, 12, and 13, in an embodiment, the first display surface 232 may be implemented in a variety of specific structural forms. For example, the first display surface 232 is configured as a concave mirror. In this arrangement, the first display surface 232 produces a magnified image of the user's face, enabling clearer observation of fine skin details during cosmetic or skincare routines.

In an embodiment, the first display surface 232 comprises a visible light camera, a first screen component, and a control module electrically connected to the power supply assembly 236. The control module is configured to drive the first screen component to display an image and to control the image size. The visible light camera may be a conventional camera utilizing visible light to capture images of the user's face. During operation, the visible light camera acquires facial images, which are processed by the control module and displayed on the first screen component for convenient viewing by the user.

In an embodiment, the outer periphery of the housing 100 corresponding to the first display surface 232 is provided with a first light-transmitting area 248. A first fill light source 240 is arranged adjacent to the first light-transmitting area 248, electrically connected to the power supply assembly 236, and can be operated by the fill light control key 234. The fill light control key 234 may be configured as a tactile push button, a capacitive touch sensor, or a multifunctional switch, enabling different modes of control such as constant illumination, stepwise brightness adjustment, or automatic dimming. In certain embodiments, the fill light control key 234 is connected to a controller, which regulates the power delivered from the power supply assembly 236 to the first fill light source 240, thereby enhancing user control and ensuring stable illumination performance. In use, the first fill light source 240 emits light through the first light-transmitting area 248 to illuminate the user's face, thereby enhancing visibility of facial details and improving the imaging clarity of the visible light camera. Additionally, the first fill light source 240 comprises a first annular light board 242 and a plurality of light-emitting elements 140 mounted on the first annular light board 242. The plurality of light-emitting elements 140 is evenly spaced along the circumference of the annular light board 242, thereby ensuring uniform illumination across the user's face.

Furthermore, the multifunctional cosmetic mirror additionally includes a first light diffuser 246 associated with the first display surface 232. The first light diffuser 246 forms the first light-transmitting area 248 and serves to improve the uniformity of the light emitted from the first fill light source 240. By diffusing the emitted light, the first light diffuser 246 prevents localized brightness and enhances the overall consistency of facial illumination.

In an embodiment, the first fill light source 240 may alternatively comprise infrared (IR) light therapy lamp beads configured to emit therapeutic light at wavelengths that are substantially invisible to the human eye. The use of IR light ensures that the phototherapy function can be performed safely without causing visual discomfort or glare to the user during operation. This enables the user to view their reflection in the first display surface 232 while simultaneously receiving skin therapy.

The infrared lamp beads may be disposed on the first annular light board 242 in a circumferential arrangement, or alternatively aggregated in clusters or distributed in a patterned configuration to concentrate therapeutic intensity on selected regions of the skin. The annular configuration promotes uniform IR light distribution across the facial surface, while clustered arrangements can be used to enhance therapeutic effects in targeted zones. Alternatively, the infrared lamp beads are positioned beneath the second surface. In all configurations, the integration of invisible IR light therapy within the first fill light source 240 allows the multifunctional device to combine cosmetic usability with simultaneous therapeutic benefit, thereby improving overall efficiency and user convenience.

In certain embodiments, the phototherapy module is configured to emit light in the far-infrared spectrum, which is particularly effective for promoting skin rejuvenation and stimulating deeper tissue layers while not interfering with the user's visibility during mirror use. During conventional operation of the mirror, far-infrared light is preferably applied so that the user may simultaneously perform cosmetic application and receive therapeutic benefits.

Referring to FIGS. 8, 10, 12, and 13, in an embodiment, the second display module 218 comprises a UV lamp 224, a UV camera 222, and a second screen assembly 220, all of which are electrically connected to the power supply assembly 236. During operation, for example, after the user has applied sunscreen, the UV camera 222 may be activated to capture an image of the user's face, and the captured ultraviolet imaging information is displayed on the second screen assembly 220.

Specifically, when the UV camera 222 is employed, areas of the skin without sunscreen reflect ultraviolet rays. The reflected UV rays enter the UV camera 222, where they are recorded by the image sensor, resulting in higher pixel values that appear as darker regions on the second screen assembly 220. In contrast, areas where sunscreen has been applied absorb ultraviolet rays, reducing or eliminating reflected UV light. Consequently, the corresponding regions on the second screen assembly 220 exhibit lower pixel values, appearing lighter in the displayed image. The degree of darkness or lightness in the image correlates with the thickness of sunscreen application: thicker sunscreen produces stronger UV absorption and darker image regions, while thinner sunscreen produces weaker UV absorption and lighter image regions. This enables the user to evaluate the uniformity and adequacy of sunscreen application. The UV lamp 224 may be activated under low-UV environments, such as indoors, to provide sufficient illumination for effective ultraviolet imaging.

In an embodiment, the power supply assembly 236 comprises a driver board 144 and a battery 150 electrically connected thereto. The driver board 144 is installed within the housing 100 and is electrically connected to the components of the second display module 218. In this configuration, the battery 150 supplies power to the driver board 144, which in turn drives the second display module 218. This arrangement eliminates the constraints of external power cords and enhances the portability and convenience of the multifunctional cosmetic mirror.

In an embodiment, the second display module 218 further includes a plane mirror 124 mounted on the light-emitting side of the second screen assembly 220. When the second screen assembly 220 is deactivated, the plane mirror 124 reflects visible light and serves as a conventional cosmetic mirror. When the second screen assembly 220 is activated, images displayed on the second screen assembly 220 are visible through the plane mirror 124, which is configured to be light-transmissive to the displayed image. Thus, the device allows dual use: as an electronic display when activated and as a traditional mirror when deactivated. In alternative embodiments, the second display module 218 may be implemented as a mirror-display module that integrates reflective and display functions, thereby eliminating the need for a separate plane mirror 124 and simplifying the device structure.

In an embodiment, the periphery of the housing 100 corresponding to the second display module 218 is provided with a second light-transmitting area 252. A second fill light assembly 142 is mounted adjacent to the second light-transmitting area 252 and is electrically connected to the power supply assembly 236. In use, the second fill light assembly 142 emits light through the second light-transmitting area 252 to provide facial illumination. This enhances the visibility of facial features during ultraviolet imaging and improves the accuracy of sunscreen coverage evaluation.

Additionally, the second fill light assembly 142 comprises a second annular light board 244 and a plurality of lamp beads 148 mounted thereon. The plurality of lamp beads 148 is evenly distributed along the circumference of the second annular light board 244 at uniform intervals, thereby ensuring consistent illumination and improving the uniformity of fill light during ultraviolet imaging.

In an embodiment, the multifunctional cosmetic mirror further includes a second light diffuser 250 arranged around the second display module 218. The second light diffuser 250 defines the second light-transmitting area 252, which diffuses the light emitted by the second fill light assembly 142. This configuration enhances the uniformity of illumination and prevents uneven brightness, thereby improving both visual comfort and imaging quality.

In an embodiment, the multifunctional cosmetic mirror further comprises a stand 202, on which the housing 100 is rotatably mounted. Specifically, the stand 202 includes a bracket 206, a base plate 204, and the housing 100 is pivotally connected to the bracket 206 about a horizontal rotation axis, and the bracket 206 is mounted on the base plate 204. This arrangement permits the housing 100 to be rotated and flipped, enabling the user to selectively position either the first display module 232 or the second display module 218 for use. Such adjustability improves operational convenience and expands the functional usability of the device.

In an embodiment, the battery 150 is housed within the stand 202 or alternatively within the housing 100.

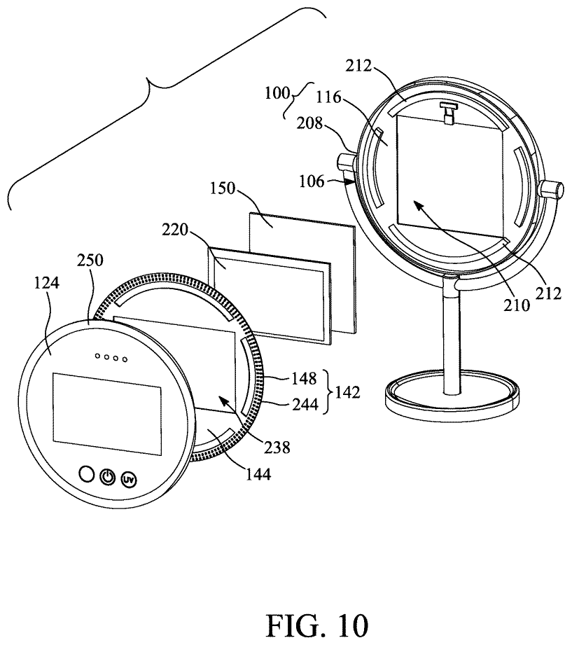

Referring to FIGS. 10 to 13, in an embodiment, the housing 100 comprises an annular wall 208 and a partition 116. The annular wall 208 is mounted on the stand 202. The mounting cavity 106 is defined between one side of the partition 116 and the annular wall 208, while a second mounting groove 216 is defined between the opposite side of the partition 116 and the annular wall 208. The partition 116 further defines a through hole 210. The second display module 218 is disposed within the mounting cavity 106, and the first display surface 232 is disposed within the second mounting groove 216. The battery 150 is at least partially accommodated within the through hole 210 of the partition 116. This arrangement reinforces the structural strength of the housing 100, provides reliable support for the first display surface 232 and the second display module 218, and optimizes use of the internal space in the thickness direction of the partition 116. By enabling partial overlap of the battery 150 with the partition 116, the overall thickness of the mirror body is reduced compared to configurations in which the battery is positioned entirely on one side of the partition. This compact structure improves the portability and user convenience of the multifunctional cosmetic mirror.

In an embodiment, the multifunctional cosmetic mirror further includes a charging component 152 disposed on the stand 202. The charging component 152 is electrically connected to the driver board 144 and is configured to connect to an external power source for recharging the battery 150. This configuration provides convenient recharging functionality while maintaining the device's portability and usability.

In this configuration, the charging component 152 is disposed on the stand 202, thereby simplifying the structure of the housing 100 and reducing the overall thickness of the mirror body. The charging component 152 may be implemented in various forms. In one embodiment, the charging component 152 is a charging connector, such as a Micro-USB female interface or a Type-C female connector, which provides a simple structure and reduces manufacturing cost. In other embodiments, the charging component 152 is a wireless charging coil, which eliminates the need for an exposed opening on the housing, thereby improving dust-proofing and water resistance of the device.

In an embodiment, the driver board 144 is formed with a mounting hole 522, into which the second screen assembly 220 is positioned. The mounting hole 238 is arranged in correspondence with the battery 150, such that the second screen assembly 220 overlaps with the driver board 144 in the thickness direction of the partition 116. This overlapping configuration further optimizes the use of internal space and increases the compactness of the housing 100.

In another embodiment, the partition 116 is reinforced by a first rib 212. The first rib 212 is arranged within the mounting cavity 106 and surrounds the avoidance through hole 210. The top surface of the first rib 212 is positioned below the notch edge of the mounting cavity 106, such that the back surface of the plane mirror 124 is supported by the first rib 212. This provides reliable installation support for the plane mirror 124 and strengthens the structural integrity of the partition 116. Optionally, the first rib 212 is spaced apart from the annular wall 208, with the second annular light board 244 disposed in the interval between the first rib 212 and the annular wall 208.

In an embodiment, the multifunctional cosmetic mirror further comprises a distance sensor 226 electrically connected to the battery 150. The distance sensor 226 detects the distance between the user and the second display module 218 and may be employed to automatically control the operation of the UV camera 222, the UV lamp 224, and/or the fill light assemblies. The distance sensor 226 may also regulate the light intensity in response to user proximity. Preferably, the distance sensor 226 is positioned above the second screen assembly 220 to enhance detection accuracy.

The device may further be configured to dynamically adjust the spectral range and intensity of emitted radiation based on the spacing between the user and the mirror, as determined by the distance sensor. For example, when the user is positioned closer to the mirror, the control circuit may automatically reduce the emission intensity or switch the emission to near-infrared wavelengths to ensure safe exposure. Conversely, when the user is located at a greater distance, the control circuit may allow higher intensity or switch to far-infrared output to maintain therapeutic efficacy. This adaptive control of wavelength and intensity provides a personalized and safe phototherapy experience while maximizing treatment effectiveness.

In an embodiment, the multifunctional cosmetic mirror comprises one or more control keys electrically connected to the battery 150. The control keys are arranged beneath the second screen assembly 220 for convenient user operation. The control keys may include, for example, a fill light control key 228 configured to control operation of the first fill light source, a key assembly 154, and at least one screen control key 230 for controlling the second screen assembly 220, such as adjusting its brightness or other display parameters. The arrangement of multiple dedicated control keys improves operational convenience and allows the user to easily manage different functional modules of the device.

In an embodiment, the partition 116 is further provided with a second rib 214. The second rib 214 is arranged within the second mounting groove 216 and is disposed around the avoidance through hole 210. The top surface of the second rib 214 is positioned below the notch edge of the second mounting groove 216, such that the back surface of the first display surface 232 abuts against the top surface of the second rib 214. This configuration provides reliable structural support for the first display surface 232, ensuring secure installation and stable operation. At the same time, the second rib 214 reinforces the partition 116, thereby improving the overall mechanical strength of the housing 100.

Optionally, the second rib 214 is spaced apart from the annular wall 208, and the first annular light board 242 is disposed in the interval between the second rib 214 and the annular wall 208. This arrangement facilitates the stable positioning of the first annular light board 242 while maintaining a compact structural layout.

In an embodiment, the multifunctional cosmetic device is provided with a rotation-based control mechanism that selectively activates the functional modules corresponding to the side of the device facing the user. The device includes a hinge or rotational shaft that permits the mirror housing to be rotated between a first orientation, in which a first functional surface faces the user, and a second orientation, in which the opposite functional surface faces the user.

The control mechanism may include a mechanical rotary switch or an orientation sensor, such as a gyroscopic sensor, accelerometer, or magnetic positioning sensor, which detects the angular position of the device relative to the hinge or shaft. When the device is rotated such that the first side is oriented toward the user, the system automatically powers on the functional modules of that side (for example, the visible light display and associated light therapy assembly), while simultaneously deactivating the modules of the opposite side. Conversely, when the device is rotated to bring the second side toward the user, the second side modules (for example, the UV imaging unit and UV fill light assembly) are activated, and the first side is powered off.

This automatic switching arrangement eliminates the need for manual toggling between the two sides, simplifies user operation, and enhances power efficiency by ensuring that only the active surface consumes energy at any given time. Furthermore, the integration of mechanical or sensor-based detection ensures reliable operation and seamless transition between different functional modes during daily use.

In certain embodiments, the multifunctional cosmetic device is configured for wireless connectivity with an external mobile application via Bluetooth, Wi-Fi, or other communication protocols. The mobile application may serve as a user interface for controlling operational modes of the device, including adjustment of infrared light intensity, duration of phototherapy sessions, switching between visible and UV imaging, and retrieval of stored image data. The application may further provide analysis algorithms that process captured images to generate diagnostic insights, treatment recommendations, or progress tracking reports. Cloud connectivity may optionally be provided for remote data storage, multi-device synchronization, or integration with professional dermatological consultation services.

The device may further support individualized user profiles stored either locally within the control unit or in association with the connected mobile application. Each profile may include parameters such as skin type, treatment history, preferred illumination settings, and captured imaging records. Upon identification of a user, the device can automatically retrieve the corresponding profile and configure operational parameters accordingly, thereby enabling personalized skincare analysis and therapy. Profile data may also be used to generate time-based progress logs, reminders for treatment cycles, or comparative imaging reports, thereby enhancing long-term usability and user engagement.

The present invention provides a multifunctional cosmetic device that integrates a mirror surface, a phototherapy module, and an ultraviolet imaging system into a single compact housing, thereby combining cosmetic, diagnostic, and therapeutic functions. Owing to its dual-faced configuration, the device can be conveniently employed in the fields of personal care, skincare, cosmetics, and dermatology. Users may perform routine grooming and makeup application while simultaneously receiving therapeutic light treatment, or evaluate sunscreen coverage and skin conditions using the UV imaging module. The device is suitable for both home use and professional environments such as beauty salons, dermatological clinics, and cosmetic retail outlets. By reducing the need for multiple separate instruments, the invention improves convenience, portability, and cost-effectiveness, thereby demonstrating significant industrial applicability across consumer and professional markets.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A multifunctional cosmetic mirror device comprising:
a mirror frame;
a mirror surface configured for use as a cosmetic mirror mounted on a first side of the mirror frame;
at least one module mounted on a second side of the mirror frame; and
wherein the at least one module is selected from an imaging module configured to analyze a skin condition of a user, or a phototherapy module configured to apply therapeutic light to a user's skin.

2. The multifunctional cosmetic mirror device of claim 1, wherein the device further comprises a control unit configured to selectively activate the imaging module or the phototherapy module.

3. The multifunctional cosmetic mirror device of claim 1, wherein the at least one module comprises both the imaging module and the phototherapy module.

4. The multifunctional cosmetic mirror device of claim 1, further comprising a distance sensor configured to measure a distance between the user's skin and the at least one module.

5. The multifunctional cosmetic mirror device of claim 1, further comprising a fill-light module arranged circumferentially around the mirror surface.

6. The multifunctional cosmetic mirror device of claim 1, wherein the phototherapy module is provided on the first side of the mirror frame having the mirror surface.

7. The multifunctional cosmetic mirror device of claim 6, wherein the phototherapy module is arranged on a side of the mirror surface, behind the mirror surface, or on or adjacent to the mirror surface.

8. The multifunctional cosmetic mirror device of claim 7, wherein the phototherapy module is configured to irradiate infrared light towards the user while the mirror surface is used for cosmetic viewing.

9. A multifunctional cosmetic mirror device comprising:
a frame having a first side and a second side;
a mirror surface provided on the first side of the frame;
a first display surface on the first side of the frame to display an image of a user;
a phototherapy module comprising an infrared light-emitting assembly associated with the first side of the frame and configured to irradiate a user's skin with invisible infrared light while the mirror surface reflects the visible image of the user;
an ultraviolet (UV) imaging unit disposed on the second side of the frame; and
an activation mechanism arranged in the frame and coupled to a control circuit, the activation mechanism being configured to selectively activate either the first display surface and the infrared light-emitting assembly when the first side faces the user, or the UV imaging unit when the second side faces the user.

10. The multifunctional cosmetic mirror device of claim 9, wherein the activation mechanism comprises a mechanical limit switch actuated by rotation of the frame.

11. The multifunctional cosmetic mirror device of claim 9, wherein the activation mechanism comprises an orientation sensor configured to detect whether the first side or the second side of the frame faces the user.

12. The multifunctional cosmetic mirror device of claim 9, wherein the infrared light-emitting assembly comprises at least one infrared LED operating within a wavelength range of 700 nm to 1100 nm.

13. The multifunctional cosmetic mirror device of claim 9, wherein the control circuit is configured to prevent simultaneous activation of the UV imaging unit and the infrared light-emitting assembly.

14. The multifunctional cosmetic mirror device of claim 9, wherein the UV imaging unit comprises a UV lamp and a UV-sensitive camera configured to capture and display UV features of the user's skin.

15. The multifunctional cosmetic mirror device of claim 9, wherein the displaying of the image of the user on the first display surface and the infrared light-emitting assembly are configured to operate simultaneously when the first side faces the user.

16. A method of operating a multifunctional cosmetic mirror device, the method comprising:
providing a mirror surface on a first side of a frame;
activating an infrared light-emitting assembly disposed in association with the mirror surface;
simultaneously reflecting a visible image of a user by the mirror surface and irradiating a user's skin with invisible infrared light emitted from the infrared light-emitting assembly;
detecting, by a distance sensor, a spacing between the user and the mirror surface; and
controlling, by a control circuit coupled to the infrared light-emitting assembly and the distance sensor, operation of the infrared light-emitting assembly such that infrared irradiation is enabled or disabled based on whether the spacing falls within a predetermined safe range.

17. The method of claim 16, wherein the controlling operation of the infrared light-emitting assembly comprises disabling the infrared light when the detected spacing is below a safe minimum distance.

18. The method of claim 16, wherein the infrared light-emitting assembly comprises at least one infrared LED operating within a wavelength range of 700 nm to 1100 nm.

19. The method of claim 16, further comprising rotating the frame such that a second side having a UV imaging unit faces the user, and automatically disabling the infrared light-emitting assembly while enabling the UV imaging unit.

20. The method of claim 16, further comprising enabling simultaneous activation of the mirror surface for visible reflection and the infrared light-emitting assembly for therapeutic irradiation during cosmetic application.
